(19) United States
(12) Patent Application Publication
(10) Pub. No.: US 2018/0250487 A1
(43) Pub. Date: Sep. 6, 2018

(54) CPAP Mask Connector and Pillow System

(71) Applicants: Susan Hodges, Tallahassee, FL (US); Sherri Wagner, Surprise, AZ (US)

(72) Inventors: Susan Hodges, Tallahassee, FL (US); Sherri Wagner, Surprise, AZ (US)

(21) Appl. No.: 15/450,668

(22) Filed: Mar. 6, 2017

(51) Int. Cl. A61M 16/06; A47G 9/10

(52) U.S. Cl. CPC A61M 16/0683; A47G 9/1045

(57) ABSTRACT

A CPAP mask connector and pillow system includes a pillow positionable at the rear of a user's head, a pair of upper and lower wings extending from the pillow, a fastening system incorporated into the wings and configured to attach to and secure a CPAP mask to a user. The fastening system further includes integrated elastic segments that provide additional flexibility in the fastening system.

### BACKGROUND OF THE INVENTION

### 1. Field of the Invention

This invention relates to the field of headgear used to position and secure continuous positive airway pressure (CPAP) masks, and more particularly to a headgear system that provides improved comfort and wearability for a CPAP user.

### 2. General Background

Sleep apnea (SA) is a serious, potentially life-threatening condition that is fairly common. SA is a breathing disorder generally characterized by brief interruptions of breathing during sleep. There are two types of SA that are generally recognized. The less common type, central sleep apnea, occurs when the brain fails to send the appropriate signals to the breathing muscles to initiate respirations or when the breathing muscles do not receive such signals. Obstructive sleep apnea is the far more common type and occurs when air is prevented from flowing into or out of a person's nose or mouth, because of a temporary obstruction in the breathing pathway.

For a person with SA, the number of involuntary breathing pauses, or “apneic events,” varies considerably and may range from as low as one or two per hour to as high as over sixty per hour. These breathing pauses are almost always accompanied by snoring, although not everyone who snores is experiencing SA. These pauses are interrupted by the person waking up and then resetting the obstruction in the breathing pathway. The frequent interruptions of sleep caused by SA often lead to excessive daytime sleepiness and well as a host of other significant medical conditions.

SA occurs in all age groups and within both sexes; however, it is more common in men Within the United States, it has been estimated that SA affects 1-6% of adults and 2% of children. People most likely to have or develop SA include those who snore loudly and also who are overweight, and those who have high blood pressure or have some physical abnormality in the nose, throat or other parts of the upper respiratory airways. Additionally, SA seems to run in some families, suggesting a possible genetic basis.

Certain mechanical and structural anomalies in the respiratory airways of a sleep apnetic person cause the interruptions in breathing during sleep. In some people, SA onset occurs when the throat muscles and tongue relax during sleep and partially block the opening of the respiratory airways. When the muscles of the soft palate at the base of the tongue and the uvula relax and sag, the respiratory airway becomes blocked, making breathing labored and noisy or even stopping altogether. SA also can occur in obese people when an excess amount of tissue in the respiratory airways causes them to be narrowed. With a narrowed respiratory airway, the person continues efforts to breathe, but air cannot easily flow into or out of the nose or mouth. Unknown to the person, this results in heavy snoring, periods of no breathing and frequent arousals (causing abrupt changes from deep sleep to light sleep). Ingestion of alcohol and sleeping pills increases the frequency and duration of breathing pauses in people with SA.

During the apneic event, the person is unable to breathe in oxygen and to exhale carbon dioxide, resulting in low levels of oxygen and increased levels of carbon dioxide in the blood. The reduction in oxygen and increase in carbon dioxide alert the brain to attempt to resume breathing by causing what is termed an “arousal.” With each arousal, a signal is sent from the brain to the upper respiratory airway muscles to open the airway; breathing is eventually resumed, often with a loud snort or gasp. Frequent arousals, although necessary for breathing to restart, prevent the patient from getting enough restorative deep sleep.

As a result of the serious disturbances in their normal sleep patterns, people with SA often feel extremely sleepy during the day and, as such, their concentration and daytime performance suffer. The consequences of SA range from annoying to life-threatening. They include symptoms of depression, irritability, sexual dysfunction and learning and memory difficulties, as well as falling asleep while at work, on the phone or driving. Untreated SA patients are at least three times as likely to encounter a dangerous condition or an accident as a result of SA, such as, for example, automobile or other machinery accidents. Moreover, it has been estimated that up to fifty percent of SA patients have high blood pressure. Finally, it has recently been shown that the risk for heart attack and stroke may also be increased in those with SA.

The specific therapy for SA is tailored to the individual patient based on medical history, physical examination and the results of any polysomnographic tests done on the patient. Medications are generally not effective in the treatment of SA.

Nasal CPAP is the most common treatment for SA. In this procedure, the patient wears a mask (sometimes referred to as a CPAP sleeve or device) over the mouth and nose during sleep, and pressure from an air blower forces air through the nasal passages. The air pressure is variably adjusted so that it is just enough to prevent the throat from collapsing during sleep. The pressure is continuous and constant.

However, CPAP systems are often difficult for patients to use because they are uncomfortable and ungainly. A primary difficulty is that the mask (covering the mouth and nose) that delivers the pressurized air becomes displaced during sleep. This often occurs because the strap system used to secure the mask to a person's face is uncomfortable, thereby creating head movements by the wearer that displace the mask. A primary source of this discomfort is the strap system used to secure a typical CPAP mask to a user's face. Prior art strap systems typically included a set of two narrow straps that are positioned above and below the user's ears. Each strap wraps around the user's head, attaches to the CPAP mask, and may be adjusted to vary the tension used to secure the mask to the user's head. This arrangement often causes discomfort because the straps on the rear of the head tend to become entangled with a user's hair, causing hair pulling when a user moves, and because the straps assert too much force over a narrow area on the back of a user's head. Finally, these systems, once positioned, lack flexibility, and thus lack the ability to accommodate small fitting adjustments caused by a user's movement while asleep.

Thus, there is a need for a system that secures a CPAP mask over a user's mouth and nose in a manner that minimizes discomfort so that the CPAP user is less likely to displace the mask during sleep, and thereby more likely to obtain the benefit of the CPAP system.

### BRIEF SUMMARY OF THE INVENTION

The present invention is a strap system, with an integrated pillow system, that secures a CPAP mask to a user's face. The pillow portion includes four wings (an upper pair and a lower pair) that extend from the central pillow portion. These wings preferably include hook and loop fastening systems that allow an adjustable attachment to a standard CPAP mask. The wings are positioned above and below a user's ears, and further include a shock absorbing section that allows for minor variations in fit without repositioning of the hook and loop fastening system. The pillow portion is positioned at the rear of a user's head and serves to distribute the force from the hook and loop fastening system over a broader area of the user's head, thereby reducing the discomfort associated with a CPAP mask attachment. The portions of the strap system and pillow system in contact with the user's skin are made from a soft fabric such as a cotton/polyester blend fabric.

### BRIEF DESCRIPTION OF THE DRAWINGS

For a more complete understanding of the present invention and the advantages thereof, reference is now made to the following description taken in conjunction with the accompanying drawings in which like reference numbers indicate like features and wherein:

FIG. 1 is a side view of the present invention attached to a user;

FIG. 2 is a plan view of an implementation of the present invention in its fully extended state;

FIG. 3 is a plan view of an implementation of the present invention in its fully extended state;

### REFERENCE NUMBERS INCLUDED IN THE DRAWINGS

The below table provides a ready reference for the reference numbers used in the appended drawings and in this specification:

| Ref. No. | Description | Ref. No. | Description |
|---|---|---|---|
| 100 | Pillow system | 101 | CPAP mask |
| 102 | User | 201 | Hook fastener |
| 202 | Loop fastener | 203 | Elastic |
| 204 | Upper Wing | 205 | Central pillow |
| 206 | Lower wing | 301 | Zipper |
| 207 | User's Ear | | |

### DETAILED DESCRIPTION OF THE INVENTION THROUGH THE DRAWINGS

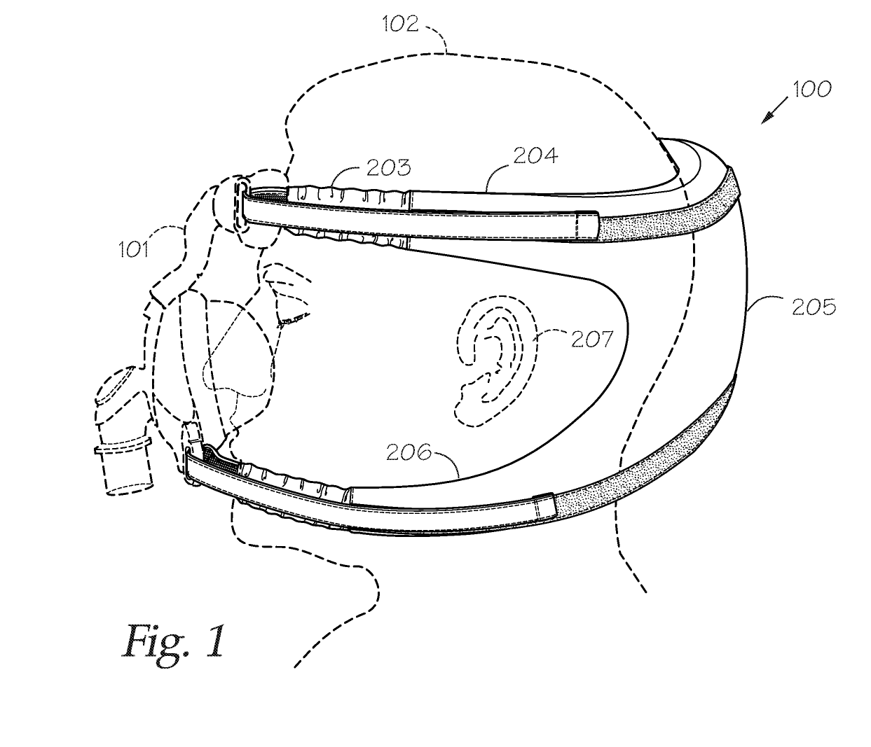

Referring to FIG. 1, the pillow system 100 of the present invention is shown attached to a user 102. Referring to FIG. 2, Pillow System 100 includes a central pillow portion 205. Extending from the central pillow portion 205 are a pair of upper wings 204 and a pair of lower wings 206. As shown in FIG. 1, in use, the central pillow portion 205 is positioned at the rear of user's 102 head, and upper wings 204 are positioned above user's ear 207 and lower wings 206 are positioned below user's ear 207.

Referring back to FIG. 2, attached to each distal end of upper wings 204 and lower wings 206 is a section of elastic 203. Preferably, each section of elastic 203 has a width of ¾ inch and a length of ¾ inches. Elastic 203 provides a flexible connection between upper and lower wings 204 and 206 and hook fasteners 201. Attached to each distal end of each section of elastic 203 is a length of hook fastener 201. Preferably, hook fastener 201 is a strap of about ½ inch width and about 7 inches in length. A pair of loop fasteners 202 are attached to the upper and lower portions of pillow system 100 and extend across central pillow 205 and to each end of upper wings 204 and lower wings 206. Preferably, loop fasteners 202 are straps of about ½ inch in width and about 16 inches in length. Alternatively, the hook and loop fastener components 201 and 202 of this system may be interchanged. Further, alternative fastener system using buttons, straps, or other connectors may be used. This description is not intended to limit the type of fastening systems that may be employed to achieve the desired function.

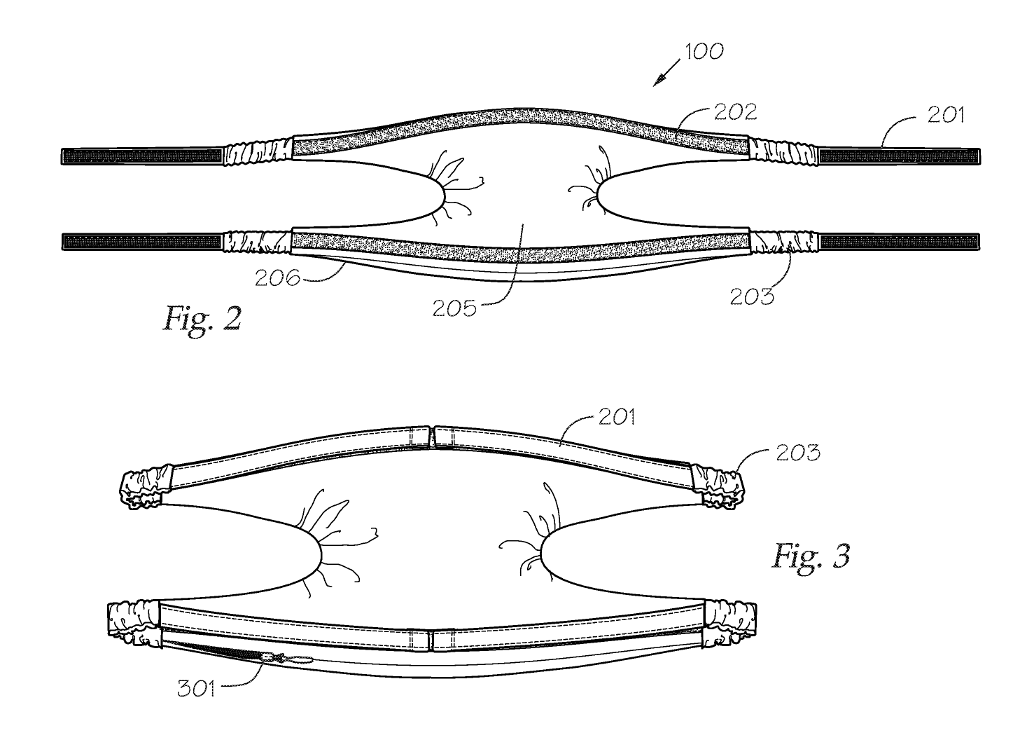

Referring to FIG. 3, hook fastener sections 201 are shown engaged with loop fastener sections 202. Zipper 301 is positioned at the bottom of pillow system 100 and allows access to the interior of pillow system 100. Preferably, about 50 g of hypo allergenic polyester fill (not shown) is placed through zipper 301 into the interior of central pillow 205. Alternative fills such as foam pads may also be utilized. This fill may be removed allowing for hand or machine washing of pillow system 100. Regular washing greatly improves usability and associated user comfort. Alternatively, pillow system 100 may be constructed without zipper access, or access may be provided by other means including flaps, buttons, and hook and loop fasteners.

In use, as shown in FIG. 1, the pillow system 100 of the present invention offers many advantages. First, the upper and lower wings 204 and 206, and their associated hook fasteners 201 and loop fasteners 202, facilitate the adjustable attachment of pillow system 100 to CPAP mask 101 in a manner that minimizes facial coverage and thus minimizes claustrophobic effects. Second, the portions of pillow system 100 in contact with a user 102 are constructed of a soft polyester/cotton blend fabric that minimizes chaffing and wicks perspiration. Third, the central pillow 205 distributes the force caused by securing pillow system 100 to CPAP mask 101 such that pressure points on the rear of user's head 102 is avoided. Fourth, once a user 102 has attached pillow system 100 to CPAP mask 101, elastic sections 203 allow user 102 to make movements while sleeping that temporarily alter the tension levels in the fastener systems without causing the CPAP mask 101 to slip or slide or otherwise become mal-positioned.

As a result, pillow system 100 provides a secure, comfortable, and reliable way to secure CPAP mask 101 to a user 102.

What is claimed is:

1. A pillow system for attaching a CPAP mask comprising:
a. a pillow having an upper section and a lower section;
b. a pair of upper wings extending from said pillow upper section;
c. a pair of lower wings extending from said pillow lower section; and
d. a fastening system attached to said pillow;

2. The pillow system of claim 1 further including:
a. a pair of elastic members attached to said pair of upper wings; and
b. a pair of elastic members attached to said pair of lower wings.

3. The pillow system of claim 2 wherein:
a. said elastic members attached to said pair of upper wings are integrated into said fastening system; and
b. said elastic members attached to said pair of lower wings are integrated into said fastening system.

4. A pillow system for attaching a CPAP mask comprising:
a. a pillow;
b. a fastening system attached to said pillow, wherein said fastening system is configured to secure a CPAP mask to a user's face;
c. an elastic connector integrated into said fastening system.

5. The pillow system of claim 4 wherein said pillow further includes a pair of upper wings and a pair of lower wings.

6. The pillow system of claim 5 wherein said pillow further includes removable pillow fill.

7. The pillow system of claim 5 wherein said fastening system is a hook and loop fastening system.

8. A pillow system for attaching a CPAP mask comprising:
a. a pillow, said pillow having a central portion and a pair of upper wings and a pair of lower wings extending from a top and a bottom of said central portion, said pillow configured to rest against a rear of a user's head;
b. an upper fastening system having a first portion attached to said pillow and extending across said upper wings and across said central portion;
c. a lower fastening system having a first portion attached to said pillow and extending across said lower wings and across said central portion;
d. a pair of elastic connectors attached to a pair of ends of said upper wings;
e. a pair of elastic connectors attached to a pair of ends of said lower wings;
f. said upper fastening system further including a pair of second portions attached to said elastic connectors attached to said upper wings; and
g. said lower fastening system further including a pair of second portions attached to said elastic connectors attached to said lower wings.

9. The pillow system of claim 8 wherein said fastening system is a hook and loop fastening system.
